(19) United States
(12) Patent Application Publication
(10) Pub. No.: US 2016/0174809 A1
(43) Pub. Date: Jun. 23, 2016

(54) Robust Storage and Transmission of Capsule Images

(71) Applicant: CAPSO VISION, INC., Saratoga, CA (US)

(72) Inventor: Kang-Huai Wang, Saratoga, CA (US)

(21) Appl. No.: 14/909,539

(22) PCT Filed: Oct. 3, 2013
(86) PCT No.: PCT/US13/63327

§ 371 Date: Feb. 2, 2016

(51) Int. Cl. A61B 1/00; A61B 1/06; A61B 1/04

(52) U.S. Cl. CPC A61B 1/00009; A61B 1/041; A61B 1/0661; A61B 1/00016

(57) ABSTRACT

The present invention discloses reliable image storage for a capsule camera device comprising a light source, an image sensor, an archival memory and a processing module within a housing. The processing module is configured to store the image frames in the archival memory so that any N consecutive image frames are stored in non-contiguous memory areas of the archival memory or to transmit any N consecutive image frames in separate wireless channels. In one embodiment, the processing module is configured to generate a first sequence and a second sequence from the image frames, and to store the first sequence in a first memory area of the archival memory or transmit in a first wireless channel, and to store the second sequence in a second memory area of the archival memory or to transmit in a second wireless channel.

### CROSS REFERENCE TO RELATED APPLICATIONS

The present invention is related to U.S. Pat. No. 7,983,458, entitled “in vivo Autonomous Camera with On-Board Data Storage or Digital Wireless Transmission in Regulatory Approved Band”, granted on Jul. 19, 2011 and to U.S. Pat. No. 7,817,354, entitled “Panoramic Imaging System”, granted on Oct. 19, 2010. These U.S. patents are hereby incorporated by reference in their entireties.

### FIELD OF THE INVENTION

The present invention relates to diagnostic imaging inside the human body. In particular, the present invention relates to system and method for protecting archived image frames and other data captured by the capsule device.

### BACKGROUND AND RELATED ART

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer the image data as an electrical signal to the proximal end through a cable. Endoscopes allow a physician control over the field of view and are well-accepted diagnostic tools. However, they do have a number of limitations, present risks to the patient, are invasive and uncomfortable for the patient, and their cost restricts their application as routine health-screening tools.

Because of the difficulty traversing a convoluted passage, endoscopes cannot reach the majority of the small intestine and special techniques and precautions, that add cost, are required to reach the entirety of the colon. Endoscopic risks include the possible perforation of the bodily organs traversed and complications arising from anesthesia. Moreover, a trade-off must be made between patient pain during the procedure and the health risks and post-procedural down time associated with anesthesia. Endoscopies are necessarily inpatient services that involve a significant amount of time from clinicians and thus are costly.

An alternative in vivo image sensor that addresses many of these problems is capsule endoscope. A camera is housed in a swallowable capsule, along with a radio transmitter for transmitting data, primarily comprising images recorded by the digital camera, to a base-station receiver or transceiver and data recorder outside the body. The capsule may also include a radio receiver for receiving instructions or other data from a base-station transmitter. Instead of radio-frequency transmission, lower-frequency electromagnetic signals may be used. Power may be supplied inductively from an external inductor to an internal inductor within the capsule or from a battery within the capsule.

An autonomous capsule camera system with on-board data storage was disclosed in the U.S. Pat. No. 7,983,458, entitled “In Vivo Autonomous Camera with On-Board Data Storage or Digital Wireless Transmission in Regulatory Approved Band,” granted on Jul. 19, 2011. This patent describes a capsule system using on-board storage such as semiconductor nonvolatile archival memory to store captured images. After the capsule passes from the body, it is retrieved. Capsule housing is opened and the images stored are transferred to a computer workstation for storage and analysis. For capsule images either received through wireless transmission or retrieved from on-board storage, the images will have to be displayed and examined by diagnostician to identify potential anomalies.

FIG. 1 illustrates an exemplary capsule system with on-board storage. The capsule system 110 includes illuminating system 12A and a camera that includes optical system 14A and image sensor 16. A semiconductor nonvolatile archival memory 20 may be provided to allow the images to be stored and later retrieved at a docking station outside the body, after the capsule is recovered. System 110 includes battery power supply 24 and an output port 26. Capsule system 110 may be propelled through the GI tract by peristalsis.

Illuminating system 12A may be implemented by LEDs. In FIG. 1, the LEDs are located adjacent to the camera's aperture, although other configurations are possible. The light source may also be provided, for example, behind the aperture. Other light sources, such as laser diodes, may also be used. Alternatively, white light sources or a combination of two or more narrow-wavelength-band sources may also be used. White LEDs are available that may include a blue LED or a violet LED, along with phosphorescent materials that are excited by the LED light to emit light at longer wavelengths. The portion of capsule housing 10 that allows light to pass through may be made from bio-compatible glass or polymer.

Optical system 14A, which may include multiple refractive, diffractive, or reflective lens elements, provides an image of the lumen walls on image sensor 16. Image sensor 16 may be provided by charged-coupled devices (CCD) or complementary metal-oxide-semiconductor (CMOS) type devices that convert the received light intensities into corresponding electrical signals. Image sensor 16 may have a monochromatic response or include a color filter array such that a color image may be captured (e.g. using the RGB or CYM representations). The analog signals from image sensor 16 are preferably converted into digital form to allow processing in digital form. Such conversion may be accomplished using an analog-to-digital (A/D) converter, which may be provided inside the sensor (as in the current case), or in another portion inside capsule housing 10. The A/D unit may be provided between image sensor 16 and the rest of the system. LEDs in illuminating system 12A are synchronized with the operations of image sensor 16. Processing module 22 may be used to provide processing required for the system such as image processing and video compression. The processing module may also provide needed system control such as to control the LEDs during image capture operation. The processing module may also be responsible for other functions such as managing image capture and coordinating image retrieval.

After the capsule camera traveled through the GI tract and exits from the body, the capsule camera is retrieved and the images stored in the archival memory are read out through the output port. The received images are usually transferred to a base station for processing and for a diagnostician to examine. The accuracy as well as efficiency of diagnostics is most important. A diagnostician is expected to examine all images and correctly identify all anomalies. In order to help the diagnostician to perform the examination more efficiently without compromising the quality of examination, the received images are subject to processing of the present invention by displaying multiple sub-sequences of the images in multiple viewing windows concurrently. The desire of using multiple viewing windows is not restricted to the conventional capsule camera. For capsule cameras having panoramic view, the need for efficient viewing for diagnostics also arises.

Besides the above mentioned forward-looking capsule cameras, there are other types of capsule cameras that provide side view or panoramic view. A side or reverse angle is required in order to view the tissue surface properly. Conventional devices are not able to see such surfaces, since their FOV is substantially forward looking. It is important for a physician to see all areas of these organs, as polyps or other irregularities need to be thoroughly observed for an accurate diagnosis. Since conventional capsules are unable to see the hidden areas around the ridges, irregularities may be missed, and critical diagnoses of serious medical conditions may be flawed. A camera configured to capture a panoramic image of an environment surrounding the camera is disclosed in U.S. Pat. No. 7,817,354, entitled “Panoramic Imaging System”, granted on Oct. 19, 2010. The panoramic camera is configured with a longitudinal field of view (FOV) defined by a range of view angles relative to a longitudinal axis of the capsule and a latitudinal field of view defined by a panoramic range of azimuth angles about the longitudinal axis such that the camera can capture a panoramic image covering substantially a 360° latitudinal FOV.

After autonomous capsule camera was introduced in the early 2000, the state of the art has been advanced continually. For example, the advancement in electronic technology includes, but not limited to, the ever decreasing of semiconductor feature size according to the proven Moore's law for integrating more transistors with lower power consumption and higher speed operation in a single chip. With the advancement in electronic technology, a larger number of pixels can reside in one CMOS image sensor and the processing of the increased information can be facilitated by the increasing computational power of the processor. On the other hand, the increasing amount of image and other sensing data can be stored in larger memory capacity are brought about by the scaling down of electronic feature size. The case is also true for a capsule system with external storage and the data is transmitted wirelessly from the capsule within the body.

In addition to the increase of resolution, the frame rate for capsule images also increases by leaps and bounds from a frame rate of about 2 per second in early 2000 to over 30 in peak rate in late 2000. The increased frame rate can reduce the un-imaged gaps between subsequent images so as to increase detection rate of anomaly. However, the detection rate of anomaly or any feature of interest may not increase proportionally to the increase of frame rate. The improvement in the detection rate may be diminishing, particularly at higher frame rates.

For a capsule system with on-board storage, the reliability of image data stored in the memory device is extremely crucial. The admission of a capsule camera will require substantial preparation for a patient to purge the waste in the GI tract. If the memory device fails, not only the efforts but also the money associated with the capsule device and services are wasted. Even if the memory device fails partially, the examination result will be compromised. Therefore, it is desired to improve the reliability of data storage without substantially increase system cost and/or power consumption for the capsule system with on-board storage. For a capsule system with wireless transmitter, it will face similar reliability issue. If the wireless transmission channel is affected by noises, interferences or other channel impairments, valuable images may be lost. Therefore, it is also desirable to improve the reliability of wireless transmission without substantially increase system cost and/or system power consumption for the capsule system with a wireless transmitter.

### BRIEF SUMMARY OF THE INVENTION

The present invention discloses reliable image storage for a capsule camera device comprising a light source, an image sensor, an archival memory or a wireless transmitter, and a processing module within a housing. The processing module is configured to store the image frames in the archival memory or to transmit the image frames using the wireless transmitter so that any N consecutive image frames are split in two or more non-contiguous memory areas of the archival memory or two or more separate wireless channels. Alternatively, at least one of the N consecutive image frames is stored in both the non-contiguous memory areas of the archival memory or transmitted at both of the wireless channels. N is an integer determined according to an image frame rate. In one embodiment, the processing module is configured to generate a first sequence and a second sequence from the image frames, and the first sequence is stored in a first memory area of said two or more non-contiguous memory areas or transmitted at a first wireless channel of said two or more separate channels, and the second sequence is stored in a second memory area of said two or more non-contiguous memory areas or transmitted at a second wireless channel of said two or more separate channels. The archival memory may comprise multiple chips or multiple dies, and the first memory area and the second memory area use different chips or different dies. The first sequence and the second sequence may be generated by interleaving the image frames. For example, the first sequence corresponds to odd-numbered pictures of the image frames and the second sequence corresponds to even-numbered pictures of the image frames. The two or more separate wireless channels may correspond to two or more channels at two or more different frequencies in a frequency division multiple access (FDMA) system, two or more different time slots in a time division multiple access (TDMA) system, or two or more frequency-time cells in a combined FDMA-TDMA system

In another embodiment, the processing module is configured to detect a significant image frame having significant content change from a previous image frame or having significant diagnostic feature, and to repeat the significant image frame in both the first sequence and the second sequence. The processing module may further comprise a data reduction module to reduce data storage requirement for the first sequence and the second sequence. The data reduction module can be configured to measure motion metric between two image frames. The data reduction module can also be configured to perform video compression.

### BRIEF DESCRIPTION OF THE DRAWINGS

FIG. 1 shows schematically a capsule camera system in the GI tract, where archival memory is used to store captured images to be analyzed and/or examined.

FIG. 2 illustrates an embodiment of the present invention, where the image frames are stored in two separate memory areas.

FIG. 3 illustrates another embodiment of the present invention, where the image frames are interleaved into two sequences and the two sequences are stored in two separate memory areas.

FIG. 4 illustrates another embodiment of the present invention, where a significant image frame is detected and is stored in both memory areas.

FIG. 5 illustrates another embodiment of the present invention, where the processing module comprises data reduction modules.

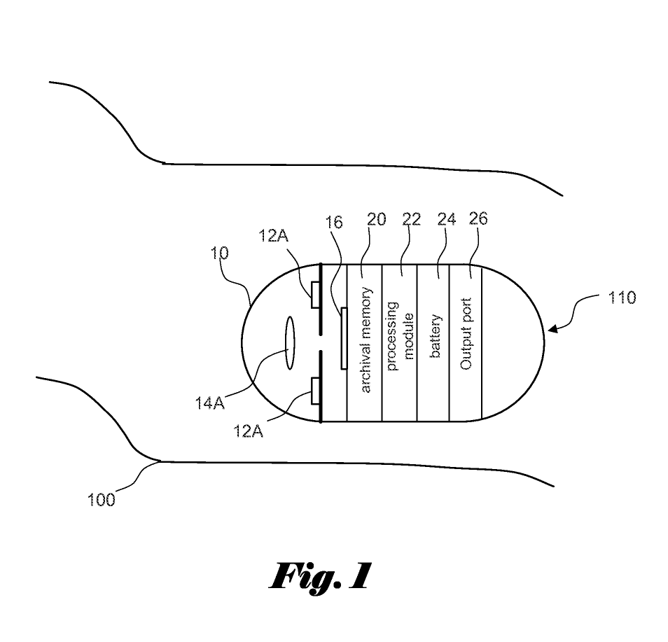

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention.

Reference throughout this specification to “one embodiment,” “an embodiment,” or similar language means that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention.

The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

The present invention discloses a system and method to increase the reliability of the image and data storage in the on-board memory or the reliability of wireless transmission. A memory chip may fully fail so that the entire memory becomes useless. A memory chip may also fail partially such as a section or a bank of memory cells fails. It is also possible that some random bits in the memory fail. When the whole memory fails, all image data becomes unavailable. However, if a section or a bank of memory fails, or a die or a chip in a multi-chip or multi-die module fails, partial image data will become unavailable. Since image data is usually stored in the memory device contiguously, partial memory failure may cause consecutive image frames unavailable. If these unavailable image frames happen to be associated with a portion of the gastrointestinal (GI) track with anomaly, the memory failure will cause a failure in detecting the anomaly. An observation of the capsule images indicates that an anomaly usually will last for multiple image frames. If the image frames are stored in separate sections, banks, chips or dies, at least some image frames in a segment of consecutive image frames may be preserved. Therefore, the anomaly may still be detected when partial memory failure occurs. Similarly, the captured image frames may be lost or damaged during wireless transmission due to noises, interferences, or other channel impairments. If the image frames are transmitted in separate wireless channels, at least some image frames in a segment of consecutive image frames may be preserved.

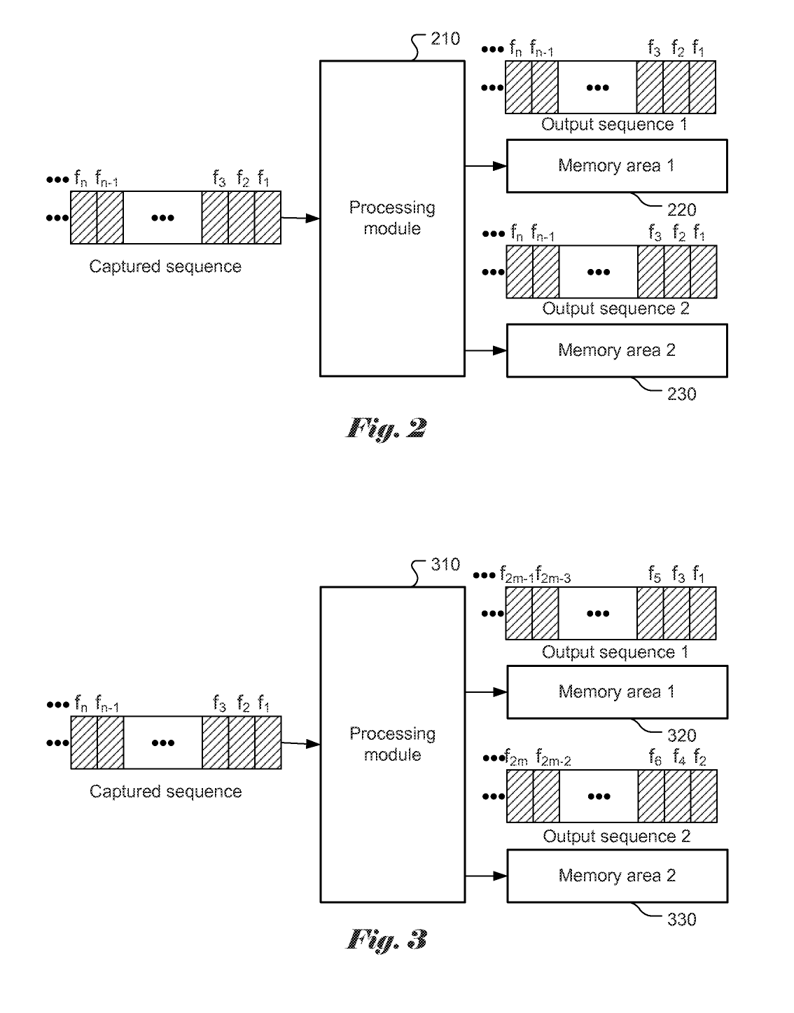

Accordingly, storing consecutive image frames in multiple non-contiguous memory areas or transmitting consecutive image frames in multiple separate wireless channels may lends a reliable means to protect image data from memory failure or transmission errors. The archival memory may also be used to store other sensing data or system data. The archival memory may correspond to a single memory chip, multiple chips or multi-chip module, or uses other chip or die packaging technology to provide required storage space with limited footage. In one embodiment, each image frame is simultaneously stored in two memory locations and preferably the two memory locations are located in two separate memory sections, banks, chips or dies as shown in FIG. 2. As shown in FIG. 2, processing module 210 is used to process the captured sequence and provides two output sequences. Output sequence 1 is stored in memory area 1 and output sequence 2 is stored in memory area 2. Memory area 1 (220) and memory area 2 (230) correspond two separate memory sections, banks, chips or dies of the archival memory. As shown in FIG. 2, both output sequence 1 and output sequence 2 correspond to the full captured sequence. In other words, FIG. 2 illustrates an example that the captured sequence is duplicated and stored in two separate memory areas. Therefore, memory failure in a contiguous (or consecutive) memory space will not cause any impact on diagnosis based on the captured sequence as long as one of the two memory areas is intact.

The embodiment illustrated in FIG. 2 implies that the memory size for the captured images will become twice as large. This may substantially increase chip cost as well as power consumption associated with data write. Furthermore, it may increase the chip footage and consequently requires a larger housing. A capsule system may not be able to afford the increase cost, power and chip size. Accordingly, another embodiment according to the present invention is illustrates in FIG. 3. As shown in FIG. 3, processing module 310 is used to process the captured sequence and provides two output sequences. Output sequence 1 is stored in memory area 1 and output sequence 2 is stored in memory area 2. Memory area 1 (320) and memory area 2 (330) correspond two separate memory sections, banks, chips or dies of the archival memory. As shown in FIG. 3, output sequence 1 and output sequence 2 correspond to odd-numbered and even numbered image frames respectively so that the total number of image frames to be stored is the same as a conventional case. Memory failure in a contiguous (or consecutive) memory space will not cause any catastrophic impact on diagnosis based on the captured sequence as long as one of the two memory areas is intact.

While FIG. 2 illustrates an example that two consecutive image frames are stored in two non-contiguous memory areas, the present invention can also be applied to cause any N consecutive image frames to be stored in two or more non-contiguous memory areas, where N is a pre-defined integer. For example, N is chosen to be 4 and the image frames are interleaved into two sequences comprising frames {1, 2, 5, 6, . . . } and frames {3, 4, 7, 8, . . . }. In another example, N is chosen to be 3 and the image frames are interleaved into three sequences comprising frames {1, 4, 7, . . . }, {2, 5, 8, . . . } and {3, 6, 9, . . . }. The selection of N can be dependent on the image frame rate. For a lower frame rate, a smaller N may be desired so that the loss of (N−1) consecutive image frames may not have big impact on the overall diagnosis. For example, if the frame rate is 2 frames per second, N may be chosen to be 2. As shown above, two or more non-contiguous memory areas can be used to store captured image frames to provide reliable storage or transmission. On the other hand, two or more separate sequences can be generated from the image frames for storing in the two or more non-contiguous memory areas. Similarly, two or more separate channels can be used to transmit the two or more separate sequences.

A side benefit of this embodiment is to relief the constraint on memory write time for the archival memory when the separate memory areas correspond to separate chips or dies with separate write ports. Since the archival memory is implemented based on non-volatile memory, the archival memory usually has a slower write time. In the system with increased image resolution and increased frame rate, the archival memory may not be able to support the high write speed required or it would increase cost of the archival memory to support the high write speed. In the example shown in FIG. 3, the write speed for each chip or die becomes half as the conventional approach. While FIG. 3 illustrates an example of interleaving the captured sequence into two output sequences for storage, the processing module may also generate more output sequences for storage in more separate memory areas.

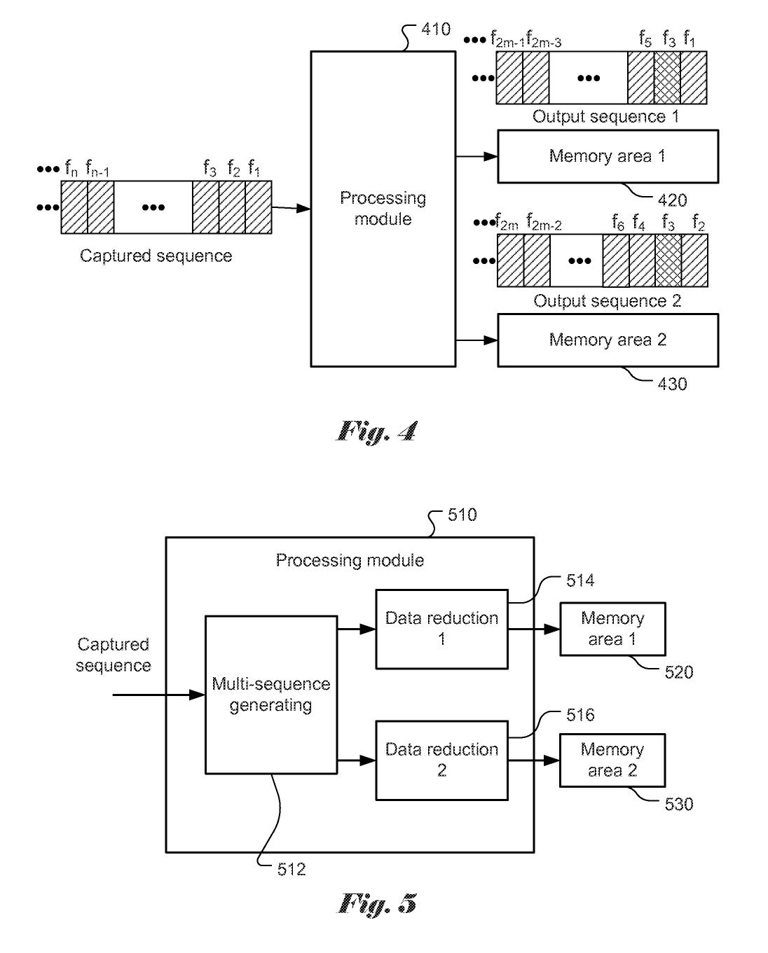

During the course of travelling through the body lumen, the capsule moves slowly as propelled by peristalsis movement. However, sometimes the capsule may undergoes quick movement momentarily and two consecutive image frames may correspond to very different scenes. If such image frames are separated into different sequences, important diagnostic features may appear in one sequence, but not in the other. Therefore, the embodiment according to FIG. 3 may still face the risk of occasionally missing detection of anomaly. Accordingly, in another embodiment of the present invention, the system detects any large change in image contents. If such large change is detected in the contents, the underlying image frame or frames are considered as “significant” and are repeated in both sequences. If any part of a sequence is lost due to memory failure, reliable anomaly detection is still possible according to this embodiment. In a capsule system, automatic feature detection may be provided for adaptive multiple sequence generation. For example, blood detection may be provided during image capture. If blood is detected for an underlying image frame, the underlying image frame is considered “significant” and is repeated in both sequences. While blood detection is used as an example, other automatic feature detection known in the field of image processing and pattern recognition may be used for adaptive multiple sequence generation. FIG. 4 illustrates an example incorporating an embodiment of the present invention. The system is similar to the system of FIG. 3. However, significant image frame or frames are identified in the system of FIG. 4. A significant image frame corresponds to an image frame having significant difference from a previous image frame or containing significant diagnostic feature as detected. For example, if image frame f₃ is detected as a significant image frame (e.g., being detected as containing blood), the processing module (410) will repeat the significant image frame (i.e., f₃) in both output sequences for storing in memory area 1 (420) and memory area 2 (430). In FIG. 4, the interleaving pattern (i.e., odd-numbered and even-numbered image frames) remains the same while a significant image frame is repeated. However, the interleaving pattern may be modified after a significant image frame is repeated. For example, after f₃ is repeated in memory area 2, the next image frame (i.e., f₄) can be stored in memory area 1, the further next image frame (i.e., f₅) can be stored in memory area 2, and so on.

During the course of travelling through the body lumen, the capsule camera may capture tens or hundreds of thousands of image frames. In order to conserve storage space, various techniques have been developed in the field to reduce required storage. For example, U.S. Pat. No. 7,983,458 discloses a technique that captures an image only when the measured motion metric exceeds a threshold. In U.S. Pat. No. 8,165,374, a motion-compensated video compression technique is applied to the image frames to substantially reduce the storage requirement at the expense of more complicated processing. Any of the data reduction techniques can be applied to the multiple sequences before the multiple sequences are stored in the separate memory areas. FIG. 5 illustrates an embodiment according to the present invention, where data reduction is applied to each of the multiple sequences. The processing module (510) includes multi-sequence generating step (512) and data reduction step (514 and 516). The data-reduced sequences are stored in memory area 1 and memory area 2 (520 and 530) separately.

After the capsule is retrieved upon its exit from the human body, the multiple sequences can be downloaded to a workstation for further processing or viewing. The multiple sequences can be rejoined into a single sequence for viewing if both sequences can be retrieved. Alternatively, each of the multiple sequences can be processed or viewed separately without rejoining. If one sequence is lost due to memory failure, the other sequence can still be used by itself for processing or viewing.

In the above examples, a system with archival memory is used as an example to illustrate robust memory storage to improve reliability of stored image frames. For a capsule system with wireless transmission, the non-contiguous memory areas are replaced by multiple wireless channels to prevent loss of N consecutive image frames. The wireless system may use frequency division multiple access (FDMA), time division multiple access (TDMA), or a combination of FDMA and TDMA to support multiple channels. For example, a capsule system may use two separate wireless channels corresponding to two different frequencies in an FDMA system to transmitted two separate image sequences. The FDMA, TDMA and FDMA-TDMA systems are well known in the field of wireless communication and the details are not repeated here.

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A capsule camera device, comprising:
a housing adapted to be swallowed, said housing enclosing:
a light source;
an image sensor for capturing image frames of a scene illuminated by the light source;
an archival memory or a wireless transmitter; and
a processing module configured to store the image frames in the archival memory or to transmit the image frames using the wireless transmitter, wherein any N consecutive image frames are split into two or more non-contiguous memory areas of the archival memory or two or more separate wireless channels, or at least one image frame is stored in at least two of said two or more non-contiguous memory areas of the archival memory or transmitted in at least two of said two or more separate wireless channels, and wherein N is a pre-defined integer.

2. The capsule camera device of claim 1, wherein the processing module is further configured to generate a first sequence and a second sequence from the image frames, and wherein the first sequence is stored in a first memory area of said two or more non-contiguous memory areas or transmitted at a first wireless channel of said two or more separate channels, and the second sequence is stored in a second memory area of said two or more non-contiguous memory areas or transmitted at a second wireless channel of said two or more separate channels.

3. The capsule camera device of claim 2, wherein the archival memory comprises multiple chips or multiple dies, and wherein the first memory area and the second memory area use different chips or different dies.

4. The capsule camera device of claim 2, wherein the first sequence and the second sequence are generated by interleaving the image frames.

5. The capsule camera device of claim 4, wherein the first sequence corresponds to odd-numbered pictures of the image frames and the second sequence corresponds to even-numbered pictures of the image frames.

6. The capsule camera device of claim 2, wherein said two or more separate wireless channels correspond to two or more channels at two or more different frequencies in a frequency division multiple access (FDMA) system, two or more different time slots in a time division multiple access (TDMA) system, or two or more frequency-time cells in a combined FDMA-TDMA system.

7. The capsule camera device of claim 2, wherein the processing module is configured to detect a significant image frame having significant content change from a previous image frame or having significant diagnostic feature, and wherein the significant image frame is repeated in the first sequence and the second sequence.

8. The capsule camera device of claim 2, wherein the processing module comprises a data reduction module to reduce data storage requirement for the first sequence and the second sequence.

9. The capsule camera device of claim 8, wherein the data reduction module is configured to measure motion metric between two image frames.

10. The capsule camera device of claim 8, wherein the data reduction module is configured to perform video compression.

11. The capsule camera device of claim 1, wherein N is determined according to an image frame rate.

12. A method for image frame storage in a capsule camera device, wherein the capsule camera device comprises a light source, an image sensor for capturing image frames of a scene illuminated by the light source, an archival memory comprising multiple chips or multiple dies or a wireless transmitter, and a processing module, the method comprising:
configuring the processing module to store the image frames in the archival memory or to transmit the image frames using the wireless transmitter, wherein any N consecutive image frames are split in two or more non-contiguous memory areas of the archival memory or two or more separate wireless channels, or at least one image frame is stored in at least two of said two or more non-contiguous memory areas of the archival memory or transmitted in at least two of said two or more separate wireless channels, and wherein N is a pre-defined integer.

13. The method of claim 12, further comprising configuring the processing module to generate a first sequence and a second sequence from the image frames by interleaving the image frames into the first sequence and the second sequence, wherein the first sequence is stored in a first memory area of said two or more non-contiguous memory areas or transmitted at a first wireless channel of said two or more separate wireless channels, and the second sequence is stored in a second memory area of said two or more non-contiguous memory areas or transmitted at a second wireless channel of said two or more separate channels.

14. The method of claim 13, wherein the first sequence corresponds to odd-numbered pictures of the image frames and the second sequence corresponds to even-numbered pictures of the image frames.

15. The method of claim 13, wherein the processing module is configured to detect a significant image frame having significant content change from a previous image frame or having significant diagnostic feature, and wherein the significant image frame is repeated in the first sequence and the second sequence.

16. The method of claim 13, wherein the processing module comprises a data reduction module to reduce data storage requirement for the first sequence and the second sequence.

17. The method of claim 16, wherein the data reduction module is configured to measure motion metric between two image frames or to perform video compression.

18. The method of claim 12, wherein N is determined according to an image frame rate.
